(19) United States
(12) Patent Application Publication
(10) Pub. No.: US 2020/0261015 A1
(43) Pub. Date: Aug. 20, 2020

(54) METHOD FOR INDEXING COGNITIVE FUNCTION

(71) Applicant: SHIMADZU CORPORATION, Kyoto-shi (JP)

(72) Inventors: Satoshi YOMOTA, Kyoto-shi (JP); Shin NAKAMURA, Kyoto-shi (JP); Kenta CHINOMI, Kyoto-shi (JP)

(73) Assignee: SHIMADZU CORPORATION, Kyoto-shi (JP)

(21) Appl. No.: 16/794,379

(22) Filed: Feb. 19, 2020

(30) Foreign Application Priority Data
Feb. 20, 2019 (JP) 2019-028294

(51) Int. Cl. A61B 5/00; A61B 5/145; A61B 5/03; G06F 17/18; G16H 10/60; G16H 50/20; G16H 50/30

(52) U.S. Cl. CPC A61B 5/4088; A61B 5/145; A61B 5/03; G16H 50/30; G16H 10/60; G16H 50/20; G06F 17/18

(57) ABSTRACT

A method for indexing cognitive function includes giving, to a subject, a work for inducing biological activity related to cognitive function, acquiring measurement data, and acquiring an index indicating the cognitive function of the subject from the measurement data of the subject using a model constructed in advance.

### CROSS-REFERENCE TO RELATED APPLICATIONS

This application claims the benefit of priority to Japanese Patent Application No. 2019-028294 filed on Feb. 20, 2019. The entire contents of this application are hereby incorporated herein by reference.

### BACKGROUND OF THE INVENTION

### Field of the Invention

The present invention relates to a method for indexing cognitive function.

### Description of the Background Art

Conventionally, a method for determining cognitive impairment is known. Such a method is disclosed in International Publication No. 2012/165602, for example.

International Publication No. 2012/165602 discloses a method for determining cognitive impairment. In this method for determining cognitive impairment, it is determined whether a subject falls under normal, mild cognitive impairment, or Alzheimer's disease using a biological signal of the brain of the subject.

Although not explicitly described in International Publication No. 2012/165602, conventionally, those who do not have Alzheimer's disease, such as non-demented persons, persons with mild cognitive impairment, or persons who are anxious about their cognitive function, may have performed interventions for dementia prevention such as exercise in order to prevent dementia. However, conventional interventions for dementia prevention could not show their effects to those who performed the interventions for dementia prevention, and thus those who performed the interventions for dementia prevention could not know the effects of the interventions for dementia prevention performed by themselves. Therefore, those who performed the interventions for dementia prevention could not be motivated to voluntarily and continuously perform the interventions for dementia prevention.

### SUMMARY OF THE INVENTION

The present invention is intended to solve the above problem. The present invention aims to provide a method for indexing cognitive function that can effectively motivate a subject to voluntarily and continuously perform interventions for dementia prevention.

In order to attain the aforementioned object, as a result of earnest investigations, the inventors have newly found that an index indicating a change in biological activity related to the cognitive function of a subject correlates with an index indicating the cognitive function of the subject between non-demented persons and persons with mild cognitive impairment. Furthermore, the inventors have newly found that there is such a correlation such that the cognitive function of the subject can be indexed based on the index indicating the change in the biological activity related to the cognitive function of the subject. A method for indexing cognitive function according to an aspect of the present invention is to index cognitive function using these new findings. That is, the method for indexing cognitive function according to this aspect of the present invention includes giving, to a subject, a work for inducing biological activity related to cognitive function, acquiring measurement data by measuring a change in the biological activity related to the cognitive function of the subject when the work is given to the subject, and acquiring an index indicating the cognitive function of the subject from the measurement data of the subject using a model constructed in advance based on the measurement data of a group of non-demented persons acquired in advance and the measurement data of a group of persons with mild cognitive impairment acquired in advance.

The method for indexing cognitive function according to this aspect of the present invention is configured as described above such that when the subject who is a non-demented person, the subject who is a person with mild cognitive impairment, the subject who is anxious about his or her cognitive function, etc. perform interventions for dementia prevention (such as exercise) to prevent dementia, the index indicating the cognitive function of the subject before and after the interventions for dementia prevention can be acquired and compared, and thus the subject can know a change in his or her cognitive function before and after the interventions for dementia prevention (i.e., the effects of the interventions for dementia prevention). In addition, when the subject continuously performs the interventions for dementia prevention, the change in the cognitive function of the subject (i.e., the effects of the interventions for dementia prevention) due to the continuous interventions for dementia prevention can be shown to the subject, and thus the subject can know the change in the cognitive function due to the continuous interventions for dementia prevention. As described above, the subject can know the effects of the interventions for dementia prevention, and thus the subject can be effectively motivated to voluntarily and continuously perform the interventions for dementia prevention. The method for indexing cognitive function according to this aspect of the present invention can be suitably implemented in a welfare facility or a sports gym, for example.

In the aforementioned method for indexing cognitive function according to this aspect, the acquiring of the index indicating the cognitive function of the subject preferably includes acquiring, as the index indicating the cognitive function of the subject, a numerical value from the measurement data of the subject using the model.

Accordingly, the effects of the interventions for dementia prevention can be more clearly measured.

In the aforementioned method for indexing cognitive function according to this aspect, the giving of the work preferably includes giving a plurality of works with different degrees of difficulty to the subject, and the acquiring of the index indicating the cognitive function of the subject preferably includes acquiring the index indicating the cognitive function of the subject from the measurement data of the subject using the model constructed based on the measurement data of the group of the non-demented persons given a same work as that given to the subject and the measurement data of the group of the persons with mild cognitive impairment given the same work as that given to the subject. Accordingly, the plurality of works with different degrees of difficulty are given such that it is possible to measure whether or not the subject has adapted to a difficult work. Consequently, the change in the biological activity related to the cognitive function of the subject can be more clearly caused, and thus the index indicating the cognitive function of the subject can be more accurately acquired.

In this case, the acquiring of the index indicating the cognitive function of the subject preferably includes acquiring the index indicating the cognitive function of the subject from the measurement data of the subject using the model constructed using a same type of feature amount as that of the measurement data of the subject, the feature amount being a difference or ratio between values of the measurement data of the subject in the plurality of works with the different degrees of difficulty.

Accordingly, the difference or ratio between the values of the measurement data of the subject is used as the feature amount such that the influence of a parameter unique to the subject, which occurs for each subject due to the shape of a measurement site of the subject, for example, can be eliminated. Therefore, the index indicating the cognitive function of the subject can be accurately acquired regardless of the subject.

In the aforementioned method for indexing cognitive function according to this aspect, the acquiring of the index indicating the cognitive function of the subject preferably includes acquiring the index indicating the cognitive function of the subject from the measurement data of the subject using the model constructed using a same type of feature amount as that of the measurement data of the subject, the feature amount being an average value of a waveform of the measurement data of the subject, a value indicating an area centroid of the waveform of the measurement data of the subject, or a value indicating a slope of the waveform of the measurement data of the subject. Accordingly, the average value of the waveform of the measurement data of the subject, the value indicating the area centroid of the waveform of the measurement data of the subject, or the value indicating the slope of the waveform of the measurement data of the subject, which is a feature amount strongly related to the cognitive function, can be used as the feature amount, and thus the index indicating the cognitive function of the subject can be accurately acquired.

In the aforementioned method for indexing cognitive function according to this aspect, the acquiring of the index indicating the cognitive function of the subject preferably includes acquiring the index indicating the cognitive function of the subject from the measurement data of the subject using the model constructed using a same type of feature amount as that of the measurement data of the subject, the feature amount being a change in an amount of oxygenated hemoglobin, a change in an amount of deoxygenated hemoglobin, or a change in a total amount of hemoglobin. Accordingly, the change in the amount of oxygenated hemoglobin, the change in the amount of deoxygenated hemoglobin, or the change in the total amount of hemoglobin that easily occurs with the work for inducing the biological activity related to the cognitive function can be used as the feature amount, and thus the index indicating the cognitive function of the subject can be accurately acquired.

In the aforementioned method for indexing cognitive function according to this aspect, the giving of the work preferably includes giving, as a task, at least one of sensory stimulation, calculation, memorization, imagination, and spatial recognition to the subject, and the acquiring of the index indicating the cognitive function of the subject preferably includes acquiring the index indicating the cognitive function of the subject from the measurement data of the subject using the model constructed based on the measurement data of the group of the non-demented persons given a same work as that given to the subject and the measurement data of the group of the persons with mild cognitive impairment given the same work as that given to the subject. Accordingly, at least one of sensory stimulation, calculation, memorization, imagination, and spatial recognition, which is a task suitable for inducing the biological activity related to the cognitive function, can be given to the subject as a task, and thus the measurement data can be acquired by reliably measuring the change in the biological activity related to the cognitive function of the subject.

In the aforementioned method for indexing cognitive function according to this aspect, the giving of the work preferably includes giving the work for inducing brain activity related to the cognitive function to the subject, and the acquiring of the measurement data preferably includes acquiring the measurement data by measuring a change in a cerebral blood flow of the subject when the work is given to the subject. Accordingly, the measurement data can be acquired by measuring the change in the cerebral blood flow suitable for measuring the cognitive function, and thus the index indicating the cognitive function of the subject can be accurately acquired.

In the aforementioned method for indexing cognitive function according to this aspect, the acquiring of the index indicating the cognitive function of the subject preferably includes acquiring the index indicating the cognitive function of the subject from the measurement data of the subject using the model including a regression model. Accordingly, the index indicating the cognitive function of the subject can be acquired from the measurement data of the subject using the model that accurately represents the correlation between the non-demented persons and the persons with mild cognitive impairment, and thus the index indicating the cognitive function of the subject can be accurately acquired.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a diagram for illustrating the overall configuration of a brain activity measurement system for implementing a method for indexing cognitive function according to an embodiment of the present invention.

FIG. 2 is a schematic view showing measurement sites at the time of measuring brain activity according to the embodiment of the present invention.

FIG. 3 is a schematic view for illustrating measurement sites at the time of measuring brain activity in accordance with the international 10-20 method.

FIG. 4 is a flowchart for illustrating the method for indexing cognitive function according to the embodiment of the present invention.

FIG. 5 is a diagram for illustrating a method for giving a work to a subject according to the embodiment of the present invention.

FIG. 6A is a diagram for illustrating a measurement data waveform according to the embodiment of the present invention.

FIG. 6B is a diagram for illustrating the area centroid of the measurement data waveform according to the embodiment of the present invention.

FIG. 6C is a diagram for illustrating the slope of the measurement data waveform according to the embodiment of the present invention.

FIG. 7 is a graph showing a correlation between a NIRS index and MCI accuracy in a regression model of ID1.

FIG. 8 is a graph showing a correlation between a NIRS index and MCI accuracy in a regression model of ID4.

FIG. 9 is a graph showing a correlation between a NIRS index and MCI accuracy in a regression model of ID7.

FIG. 10 is a graph showing a correlation between a NIRS index and MCI accuracy in a regression model of ID9.

FIG. 11 is a graph showing a correlation between a NIRS index and MCI accuracy in a regression model of ID12.

FIG. 12 is a graph showing a NIRS index distribution of evaluation data in the regression model of ID1.

FIG. 13 is a graph showing a NIRS index distribution of evaluation data in the regression model of ID4.

FIG. 14 is a graph showing a NIRS index distribution of evaluation data in the regression model of ID7.

FIG. 15 is a graph showing a NIRS index distribution of evaluation data in the regression model of ID9.

FIG. 16 is a graph showing a NIRS index distribution of evaluation data in the regression model of ID12.

### DESCRIPTION OF THE PREFERRED EMBODIMENTS

An embodiment of the present invention is hereinafter described with reference to the drawings.

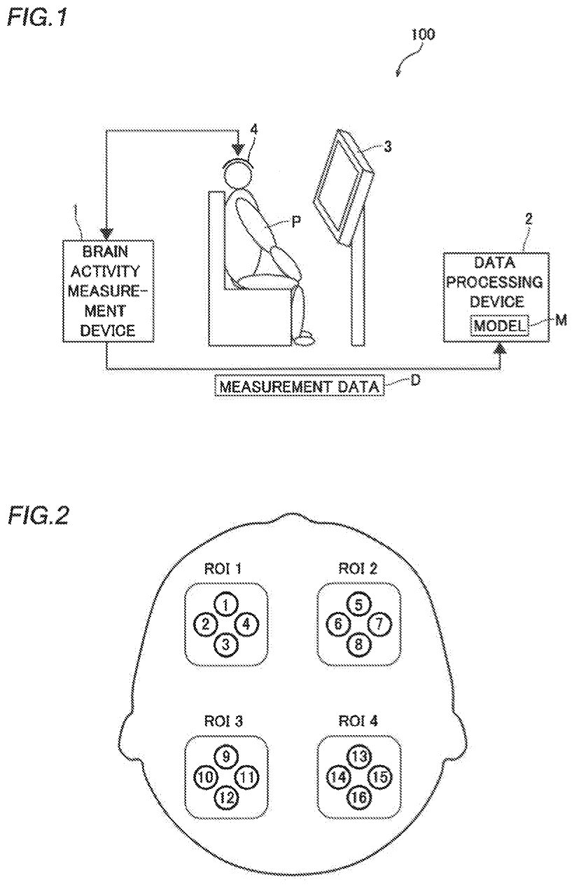

The configuration of a brain activity measurement system 100 for implementing a method for indexing cognitive function according to the embodiment of the present invention is now described with reference to FIGS. 1 to 3.

### (Configuration of Brain Activity Measurement System)

As shown in FIG. 1, the brain activity measurement system 100 includes a brain activity measurement device 1, a data processing device 2, and a display device 3.

The brain activity measurement device 1 is a device (optical measurement device) that optically measures the brain activity of a subject P using near-infrared spectroscopy (NIRS) and generates time-series measurement result data. Specifically, the brain activity measurement device 1 is a NIRS device. The brain activity measurement device 1 emits measurement light in a near-infrared wavelength region from light transmitting probes (not shown) arranged on a surface of the head of the subject P. The brain activity measurement device 1 detects the measurement light reflected in the head by causing the measurement light reflected in the head to enter light receiving probes (not shown) arranged on the surface of the head, and acquires the intensity of the measurement light (the amount of received light). A plurality of light transmitting probes and a plurality of light receiving probes are provided, and are attached to a holder 4 configured to fix each probe at a predetermined position on the surface of the head. In this embodiment, the brain activity measurement device 1 measures, as an index of a change in cerebral blood flow, the amount of change in oxygenated hemoglobin, the amount of change in deoxygenated hemoglobin, and the amount of change in total hemoglobin based on the intensity of the measurement light (the amount of received light) at a plurality of wavelengths (three wavelengths of 780 nm, 805 nm, and 830 nm, for example) and the absorption characteristics of hemoglobin.

The data processing device 2 processes measurement data D transmitted from the brain activity measurement device 1. The data processing device 2 includes a personal computer (PC) including a CPU, a memory, a hard disk drive, etc. The data processing device 2 stores in advance a model M for indexing the cognitive function of the subject P. The model M is described in detail below. The display device 3 is configured to display a work (task) to be performed by the subject P. The display device 3 is a monitor such as a liquid crystal display.

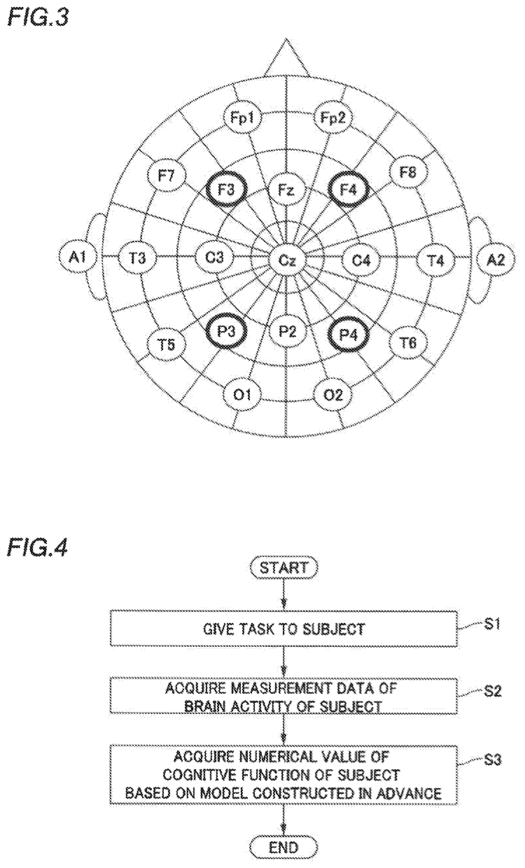

FIG. 2 shows an example of measurement sites at the time of measuring the blood flow of the brain of the subject P by the brain activity measurement device 1. FIG. 3 is a diagram showing measurement sites in accordance with the international 10-20 method. The measurement sites at the time of acquiring the measurement data D of the brain activity of the subject P shown in FIG. 2 are set within a range including F3, F4, P3 and P4 in accordance with the international 10-20 method shown in FIG. 3. Specifically, the measurement sites shown in FIG. 2 are configured as sixteen channels as shown in FIG. 2 within the range including F3, F4, P3, and P4 in accordance with the international 10-20 method. At this time, ROIs 1 to 4 are set as regions of interest (ROIs). Channels 1 to 4 of the ROI 1 are set such that the left frontal association area and the dorsolateral prefrontal area of the subject P can be measured. Channels 5 to 8 of the ROI 2 are set such that the right frontal association area and the dorsolateral prefrontal area of the subject P can be measured. Channels 9 to 12 of the ROI 3 are set such that the left somatosensory area of the subject P can be measured. Channels 13 to 16 of the ROI 4 are set such that the right somatosensory area of the subject P can be measured.

### (Method for Indexing Cognitive Function)

The method for indexing cognitive function according to this embodiment is now described with reference to FIGS. 4 to 6.

### <Step of Giving Work to Subject>

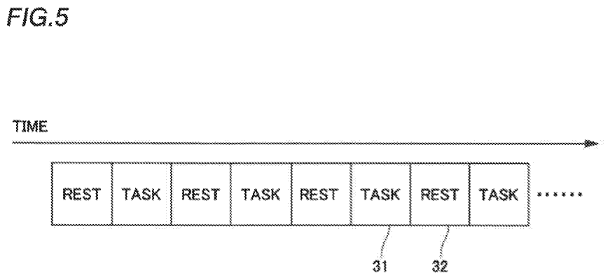

As shown in FIG. 4, the method for indexing cognitive function according to this embodiment includes a step (step S1 in FIG. 4) of giving a work (task) for inducing biological activity related to cognitive function to the subject P who desires indexing of his or her cognitive function. In this step, the subject P is given a work for inducing brain activity related to cognitive function. In this step, the subject P is given a work a plurality of times, as shown in FIG. 5. Specifically, the subject P is given a work a plurality of times such that a work period 31 in which the subject P is given a work and a rest period 32 in which the subject P is not given a work are alternately repeated. The work period 31 is 20 seconds, for example. The rest period 32 is 20 seconds, for example. In the rest period 32, a baseline for measuring a change in the cerebral blood flow of the subject P is constructed. In the rest period 32, the subject P is kept at rest, or the subject P is made to pronounce a meaningless word, for example, in order to construct the baseline. The meaningless word that the subject P pronounces during the rest period 32 is “a, i, u, e, o”, for example. Although FIG. 5 shows an example in which a task is repeated four times, the number of times the task is repeated may be other than four.

In this step, at least one of sensory stimulation, calculation, memorization, imagination, and spatial recognition is given to the subject P as a task.

Specifically, when the task to be given to the subject P is sensory stimulation, the sensory stimulation is applied to the sensory organ of the subject P. As the sensory stimulation, cold sensory stimulation by applying a cooling agent to the palm of the subject P can be used, for example. When the task to be given to the subject P is calculation, the subject P is given a calculation problem. As the calculation problem, the serial seven (100-7) used in a mini-mental state examination (MMSE) for diagnosing dementia or a revised version of the serial seven (100-7) can be used, for example. Note that the serial seven (100-7) is a problem of continuously subtracting 7 from 100. When the task to be given to the subject P is memorization and imagination, a problem in which characters with similar shapes are written on the hand of the subject P and the subject P guesses the characters is given to the subject P. The characters with similar shapes are “O”, “P”, and “Q”, for example. When the task to be given to the subject P is spatial recognition, a problem in which a landscape photograph is displayed on the display device 3 and the subject P is handed a map showing a schematic view of buildings drawn in the landscape photograph and answers where the subject P should stand to see the landscape of the landscape photograph with a number is given to the subject P.

In this step, a plurality of tasks (works) having different degrees of difficulty are given to the subject P. Specifically, the plurality of tasks having different degrees of difficulty are given to the subject P in such a manner that the degree of difficulty gradually increases. For example, when a calculation problem is given to the subject P, a problem of continuously subtracting 2 from 100 is given to the subject P in the first task, a problem of continuously subtracting 3 from 100 is given to the subject P in the second task, a problem of continuously subtracting 7 from 100 is given to the subject P in the third task, a problem of continuously subtracting 7 from 101 is given to the subject P in the fourth task, and a problem of continuously subtracting 7 from 102 is given to the subject P in the fifth task. Note that in the subtraction of an even number and the subtraction of an odd number, the degree of difficulty of the subtraction of an odd number is higher. For example, when a problem in which characters with similar shapes are written on the hand of the subject P and the subject P guesses the characters is given to the subject P, a problem in which two characters are written on the hand of the subject P is given to the subject P in the first and second tasks, and a problem in which three characters are written on the hand of the subject P is given to the subject P in the third and fourth tasks. The degree of difficulty is higher as the number of characters answered by the subject P is larger. For example, when a problem in which a landscape photograph is displayed on the display device 3 and the subject P is handed a map showing a schematic view of buildings drawn in the landscape photograph and answers where the subject P should stand to see the landscape of the landscape photograph with a number is given to the subject P, in the third and fourth tasks, the degree of difficulty of the task is increased by increasing the number of roads and buildings as compared with the first and second tasks.

### <Step of Acquiring Measurement Data>

As shown in FIG. 4, the method for indexing cognitive function according to this embodiment includes a step (step S2 in FIG. 4) of acquiring the measurement data D by measuring a change in the biological activity related to the cognitive function of the subject P when the subject P is given a work. In this step, a change in the cerebral blood flow of the subject P is measured when the subject P is given a work, and the measurement data D is acquired. Specifically, when the subject P is given a work, a change in the cerebral blood flow at each measurement site (each channel) of the subject P is measured. In this step, as an index of the change in the cerebral blood flow, a change in the amount of oxygenated hemoglobin, a change in the amount of deoxygenated hemoglobin, and a change in the total amount of hemoglobin, which is the total amount of the amount of oxygenated hemoglobin and the amount of deoxygenated hemoglobin, are measured. In this step, as shown in FIGS. 2 and 3, the change in the cerebral blood flow at each measurement site set within the range including F3, F4, P3, and P4 in accordance with the international 10-20 method is measured. In this step, as described above, the change in the cerebral blood flow at each measurement site is measured by the near-infrared spectroscopy (NIRS).

In the brain activity measurement device 1, when the change in the cerebral blood flow at each measurement site is measured, a skin blood flow in the vicinity of the measurement site at which the change in the cerebral blood flow is measured is measured simultaneously with the measurement of the change in the cerebral blood flow. Then, a value obtained by subtracting (correcting) the measured cerebral blood flow by the measured skin blood flow is used as the cerebral blood flow. Thus, when the change in the cerebral blood flow is measured, the cerebral blood flow in which the influence of the skin blood flow has been significantly reduced or prevented can be measured even when the measured cerebral blood flow includes the skin blood flow due to the skin included in an optical path of the measurement light.

### <Step of Acquiring Index Indicating Cognitive Function of Subject>

As shown in FIG. 4, the method for indexing cognitive function according to this embodiment includes a step (step S3 in FIG. 4) of acquiring an index indicating the cognitive function of the subject P from the measurement data D of the subject P using the model M (see FIG. 1) constructed in advance. In this step, a numerical value as the index indicating the cognitive function of the subject P is acquired from the measurement data D of the subject P using the model M. The model M is constructed in advance based on the measurement data D of a group of non-demented persons acquired in advance and the measurement data D of a group of persons with mild cognitive impairment acquired in advance. Specifically, the model M is constructed in advance using a regression model based on the measurement data D of the group of non-demented persons acquired in advance and the measurement data D of the group of persons with mild cognitive impairment acquired in advance.

The subject P is given the same work as a work given to the group of non-demented persons and the group of persons with mild cognitive impairment when the model M is constructed. Therefore, in this step, the index indicating the cognitive function of the subject P is acquired from the measurement data D of the subject P using the model M constructed based on the measurement data D of the group of non-demented persons given the same work as that given to the subject P and the measurement data D of the group of persons with mild cognitive impairment given the same work as that given to the subject P.

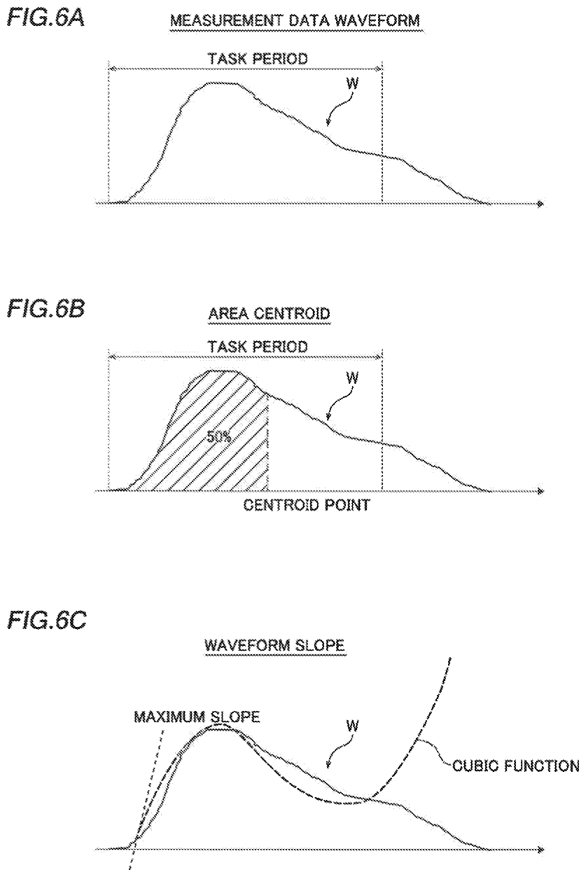

In this step, first, a feature amount for acquiring the index indicating the cognitive function is acquired from the measurement data D of the subject P. The acquired feature amount is a change in the amount of oxygenated hemoglobin, a change in the amount of deoxygenated hemoglobin, or a change in the total amount of hemoglobin, for example. Specifically, as shown in FIGS. 6A to 6C, the acquired feature amount is the average value of a waveform W of the measurement data D of the subject P, a value indicating the area centroid of the waveform W of the measurement data D of the subject P, or a value indicating the slope (a maximum value of the slope) of the waveform W of the measurement data D of the subject P in the waveform W indicating a change in each hemoglobin amount during the work period 31. More specifically, the acquired feature amount is a difference or ratio between values (the average values, the values indicating the area centroid, or the values indicating the slope) of the measurement data D of the subject P in a plurality of works with different degrees of difficulty in the change in each hemoglobin amount. That is, it is a difference or ratio between a value of the measurement data D of the subject P in a work with a low degree of difficulty and a value of the measurement data D of the subject P in a work with a higher degree of difficulty than that work. As a specific feature amount, a ratio between values indicating the area centroid of the waveform W in the change in the total amount of hemoglobin is acquired, for example.

In this step, the same type of feature amount as that acquired from the measurement data D of the group of non-demented persons and the measurement data D of the group of persons with mild cognitive impairment when the model M is constructed is acquired from the measurement data D of the subject P. Therefore, in this step, the index indicating the cognitive function of the subject P is acquired from the measurement data D of the subject P using the model M constructed using the same type of feature amount as that of the measurement data D of the subject P.

Then, in this step, an index indicating the change in the biological activity (brain activity) related to the cognitive function of the subject P is acquired from the feature amount acquired from the measurement data D. Specifically, a NIRS index, which is the index indicating the change in the biological activity (brain activity) related to the cognitive function of the subject P, is acquired from the feature amount acquired from the measurement data D using the model M constructed in advance.

Although the details are described below, as a result of earnest investigations, the inventors have newly found that there is a correlation between the NIRS index, which is the index indicating the change in the biological activity (brain activity) related to the cognitive function of the subject P, and mild cognitive impairment accuracy (hereinafter referred to as “MCI accuracy”), which is the index indicating the cognitive function of the subject P. From this finding, the inventors also have newly found that the NIRS index, which is the index indicating the change in the biological activity (brain activity) related to the cognitive function of the subject P, is acquired from the measurement data D such that the cognitive function of the subject P can be indexed.

The NIRS index is represented by the following formula (1), which is a regression model based on logistic regression, for example:

N=1/[1+exp{−(α+C0×F0+C1×F1+ . . . Cn×Fn)}] (1)

where N represents a NIRS index, a represents a constant, Cn represents a coefficient (weight), Fn represents a feature amount, and n represents a natural number.

The constant α and the coefficient Cn, which is a weight for each feature amount Fn, are values acquired in advance using the regression model in such a manner that there is a correlation between the NIRS index, which is the index indicating the change in the biological activity (brain activity) related to the cognitive function of the subject P, and the MCI accuracy, which is the index indicating the cognitive function of the subject P. Specifically, the constant α and the coefficient Cn are values acquired in advance based on the measurement data D of the group of non-demented persons and the measurement data D of the group of persons with mild cognitive impairment by assuming that a NIRS index N converges to 0 in the measurement data D of the non-demented persons and that a NIRS index N converges to 1 in the measurement data D of the persons with mild cognitive impairment. The feature amount Fn is a value acquired from the measurement data D, as described above. For example, a difference or ratio between a predetermined type of values (the average values, the values indicating the area centroids, or the values indicating the slopes) of the waveform W in a change in the amount of predetermined hemoglobin (the change in the amount of oxygenated hemoglobin, the change in the amount of deoxygenated hemoglobin, or the change in the total amount of hemoglobin) is acquired from the measurement data D for each channel or each work, and is input into the feature amount Fn.

Then, in this step, the index indicating the cognitive function of the subject P is acquired from the NIRS index acquired from the formula (1). As the index indicating the cognitive function of the subject P, the NIRS index itself may be acquired and presented to the subject P, or a score obtained by converting the NIRS index in order for the subject P to easily understand the NIRS index may be acquired and presented to the subject P. Furthermore, as the index indicating the cognitive function of the subject P, the MCI accuracy correlated with the NIRS index may be acquired and presented to the subject P. The NIRS index indicates that the larger the value, the lower the cognitive function, and the smaller the value, the higher the cognitive function.

### (Correlation Confirmation Experiment)

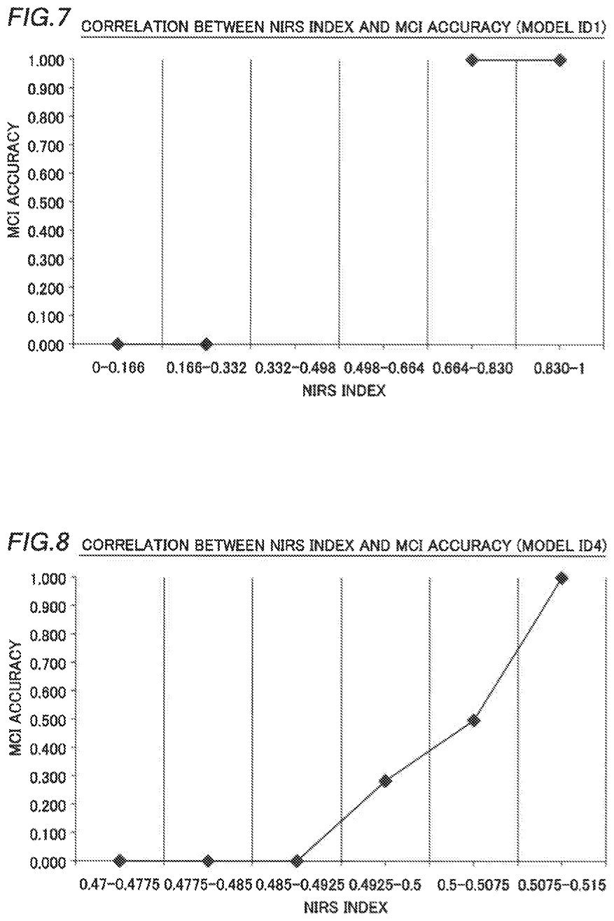

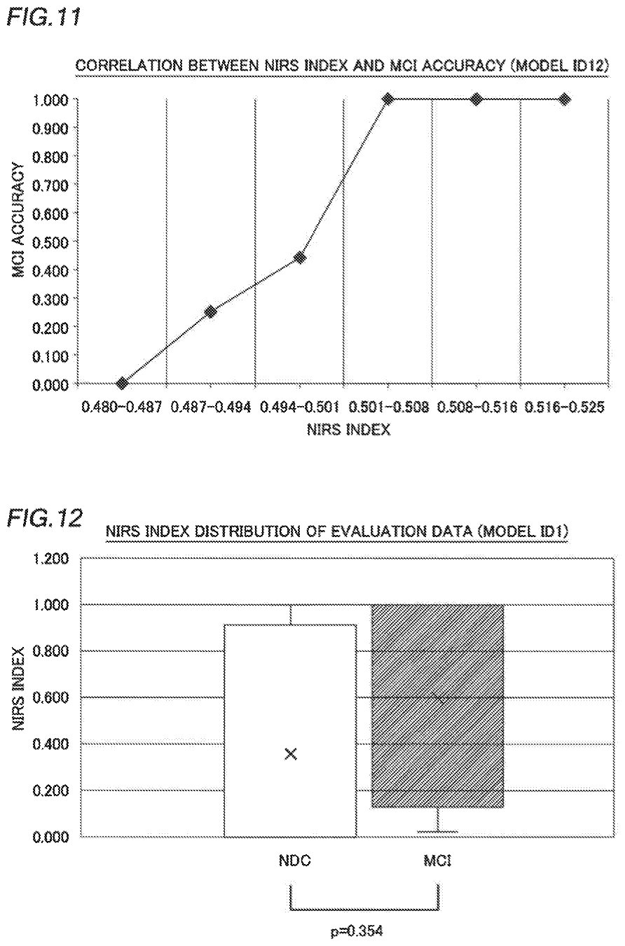

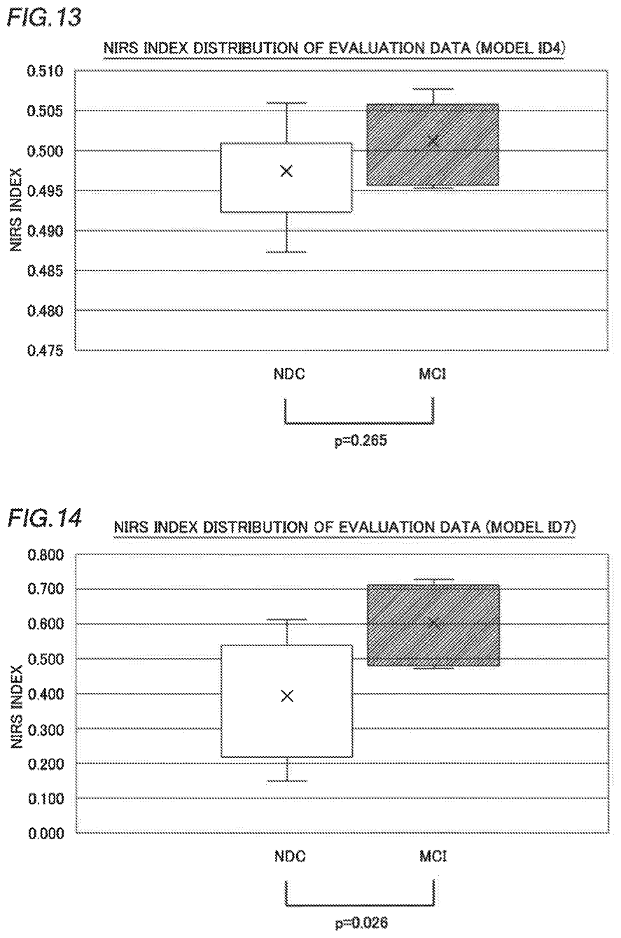

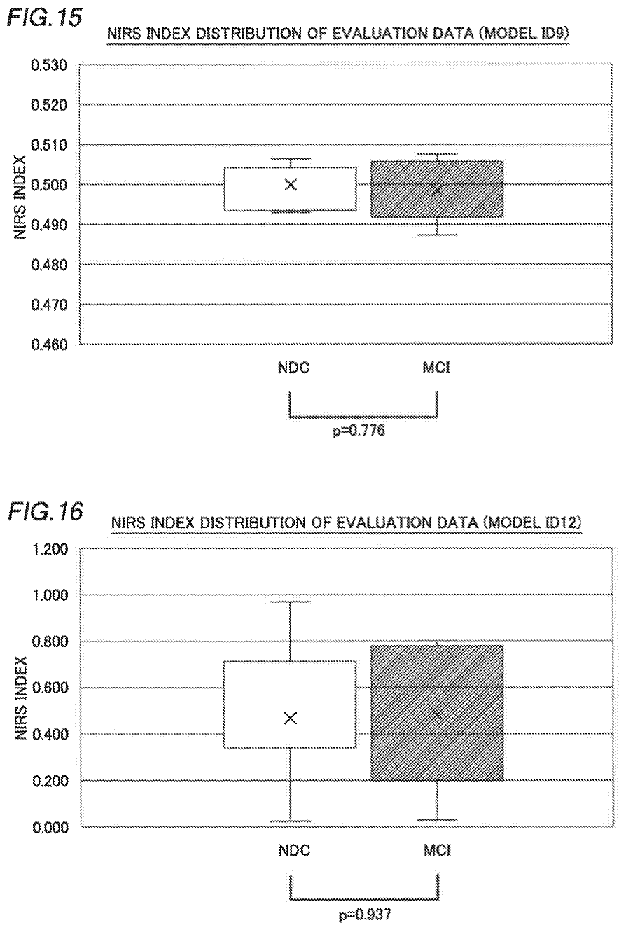

An experiment conducted in order to confirm the correlation between the NIRS index and the MCI accuracy is now described with reference to FIGS. 7 to 16.

In the experiment, as shown in TABLES 1 and 2 below, for a group of subjects including fifteen non-demented persons (a group of non-demented persons) with definitive diagnosis by a doctor and twenty-three persons with mild cognitive impairment (a group of persons with mild cognitive impairment), NIRS measurement data (hereinafter referred to as “training data” as appropriate) was acquired.

TABLE 1
TRAINING DATA (CASE LABEL: MILD
COGNITIVE IMPAIRMENT (MCI))

| | MALE | FEMALE | WHOLE |
|---|---|---|---|
| AVERAGE AGE | 70.9 | 72.8 | 71.4 |
| STANDARD | 4.42 | 5.80 | 4.42 |
| DEVIATION | | | |
| NUMBER OF | 11 | 4 | |
| PERSONS | | | |

TABLE 2
TRAINING DATA (CASE LABEL:
NON-DEMENTED CONTROL (NDC))

| | MALE | FEMALE | WHOLE |
|---|---|---|---|
| AVERAGE AGE | 69.5 | 88.6 | 69.0 |
| STANDARD | 4.63 | 5.50 | 5.29 |
| DEVIATION | | | |
| NUMBER OF | 11 | 12 | |
| PERSONS | | | |

In the experiment, the group of subjects was given a calculation task and an “OPQ” task, which are useful for monitoring a change in cognitive function, as tasks. In the calculation task, the problem of continuously subtracting 2 from 100, the problem of continuously subtracting 3 from 100, the problem of continuously subtracting 7 from 100, the problem of continuously subtracting 7 from 101, and the problem of continuously subtracting 7 from 102 were given to the subjects in this order, a task period (work period) was 20 seconds, and the rest period was 20 seconds. During the rest period, the subjects were made to pronounce the meaningless word “a, i, u, e, o” such that the baseline of the measurement data was determined. In the “OPQ” task, the problem in which two or three of the three characters of “O”, “P”, and “Q” were continuously written on the palms of the hands of the subjects P with the eyes closed, and the subjects P guessed the characters written on the palms of the hands was given to the subjects P. A total of four “OPQ” tasks including two “OPQ” tasks with two characters and two “OPQ” tasks with three characters was performed, the task period was 30 seconds, and the rest period was 40 seconds. During the rest period, the subjects were kept at rest.

In the experiment, the measurement sites were configured as the sixteen channels as shown in FIG. 2 within the range including F3, F4, P3, and P4 in accordance with the international 10-20 method as shown in FIG. 3. At this time, the ROI 1 to the ROI 4 as shown in FIG. 2 were set as regions of interest (ROI).

As shown in TABLE 3 below, twelve regression models were obtained based on the measurement data of the group of subjects obtained by the experiment. Variables (feature amounts) of the regression models were changes in two types of hemoglobin amount (the change in the amount of deoxygenated hemoglobin (deoxyHb in TABLE 3) and the change in the total amount of hemoglobin (totalHb in TABLE 3)), three types of ROI (ROIs 1, 2 and 4), and three types of waveform feature value (the average value, the value indicating the area centroid, and the value indicating the slope) used to acquire the ratio between a plurality of works with different degrees of difficulty. For example, in a regression model of ID1, the variables are the change in the amount of deoxygenated hemoglobin, the ROIs 1, 2, and 4, and the value indicating the area centroid of the waveform of the measurement data. In other words, the regression model of ID1 is obtained using, as feature amounts, ratios between values indicating the area centroids of the waveforms of the measurement data in a plurality of works with different degrees of difficulty in the changes in the amounts of deoxygenated hemoglobin for twelve channels of the ROIs 1, 2, and 4.

TABLE 3

| REGRESSION | HEMO- | SIGNAL | MEASUREMENT |
|---|---|---|---|
| MODEL ID | GLOBIN | FEATURE | ch |
| 1 | deoxyHb | AREA CENTROID | 12ch/ROI 1-2-4 |
| 2 | deoxyHb | WAVEFORM SLOPE | 12ch/ROI 1-2-4 |
| 3 | deoxyHb | AVERAGE | 12ch/ROI 1-2-4 |
| 4 | deoxyHb | AREA CENTROID | 8ch/ROI 1-4 |
| 5 | deoxyHb | WAVEFORM SLOPE | 8ch/ROI 1-4 |
| 6 | deoxyHb | AVERAGE | 8ch/ROI 1-4 |
| 7 | totalHb | AREA CENTROID | 12ch/ROI 1-2-4 |
| 8 | totalHb | WAVEFORM SLOPE | 12ch/ROI 1-2-4 |
| 9 | totalHb | AVERAGE | 12ch/ROI 1-2-4 |
| 10 | totalHb | AREA CENTROID | 8ch/ROI 1-4 |
| 11 | totalHb | WAVEFORM SLOPE | 8ch/ROI 1-4 |
| 12 | totalHb | AVERAGE | 8ch/ROI 1-4 |

Although the twelve regression models are represented by the above formula (1), different feature amounts are used, and thus different constants α and coefficients Cn are determined. Note that the constant α and the coefficient Cn of each regression model are determined, setting objective variables of the measurement data of the group of non-demented persons to 0 and objective variables of the measurement data of the group of persons with mild cognitive impairment to 1. In other words, the constant α and the coefficient Cn of each regression model are determined by calculation, assuming that the NIRS index N converges to 0 in the measurement data of the non-demented persons and that the NIRS index N converges to 1 in the measurement data D of the persons with mild cognitive impairment.

In order to evaluate the performance of the twelve regression models, as shown in TABLES 4 and 5 below, the NIRS measurement data (hereinafter referred to as “evaluation data” as appropriate) of eight non-demented persons (a group of non-demented persons) and the NIRS measurement data (hereinafter referred to as “evaluation data” as appropriate) of five persons with mild cognitive impairment (a group with persons with mild cognitive impairment) were applied to each regression model to obtain an accuracy rate. At this time, when the measurement data of the non-demented persons (persons with mild cognitive impairment) was applied to a regression model, the answer was correct when the regression model determined that the subjects P were the non-demented persons (persons with mild cognitive impairment). When the measurement data of the non-demented persons (persons with mild cognitive impairment) was applied to the regression model, the answer was incorrect when the regression model determined that the subjects P were the persons with mild cognitive impairment (non-demented persons). The accuracy rate by each regression model is as shown in TABLE 6 below.

TABLE 4
EVALUATION DATA (CASE LABEL: MILD
COGNITIVE IMPAIRMENT (MCI))

| | MALE | FEMALE | WHOLE |
|---|---|---|---|
| AVERAGE AGE | 56.3 | 69.0 | 67.4 |
| STANDARD | 5.31 | 2.00 | 4.50 |
| DEVIATION | | | |
| NUMBER OF | 3 | 2 | |
| PERSONS | | | |

TABLE 5
EVALUATION DATA (CASE LABEL:
NON-DEMENTED CONTROL (NDC))

| | MALE | FEMALE | WHOLE |
|---|---|---|---|
| AVERAGE AGE | 66.5 | 68.3 | 67.4 |
| STANDARD | 2.49 | 4.82 | 3.84 |
| DEVIATION | | | |
| NUMBER OF | 4 | 4 | |
| PERSONS | | | |

TABLE 6

| REGRES- | EVALUATION | ACCURACY | |
|---|---|---|---|
| SION | DATA | RATE | ACCURACY |
| MODEL | ACCURACY RATE | (NON-DEMENTED | RATE |
| ID | (WHOLE) | CONTROL) | (MCI) |
| 1 | 61.5% | 62.5% | 60.0% |
| 2 | 46.2% | 62.5% | 20.0% |
| 3 | 53.8% | 50.0% | 60.0% |
| 4 | 76.9% | 87.5% | 60.0% |
| 5 | 38.5% | 62.5% | 0% |
| 6 | 46.2% | 50.0% | 40.0% |
| 7 | 76.9% | 87.5% | 60.0% |
| 8 | 38.5% | 37.5% | 40.0% |
| 9 | 61.5% | 62.5% | 60.0% |
| 10 | 61.5% | 37.5% | 100% |
| 11 | 46.2% | 50.0% | 40.0% |
| 12 | 61.5% | 62.5% | 60.0% |

As shown in TABLE 6, in the five regression models of IDs 1, 4, 7, 9, and 12, a high accuracy rate of 60% or more was obtained in both cases of determining the non-demented persons and the persons with mild cognitive impairment.

In addition, for the five regression models having a high accuracy rate, a correlation between the NIRS index, which was the index indicating the change in the biological activity (brain activity) related to the cognitive function of the subject P, and the MCI accuracy, which was the index indicating the cognitive function of the subject P, was examined based on training data (measurement data of thirty-eight subjects). The results of examination of the correlation are shown as graphs in FIGS. 7 to 11. The graphs shown in FIGS. 7 to 11 show the MCI accuracy in a certain NIRS index range. For example, in FIG. 7, the MCI accuracy at each of NIRS indices in a range of 0-0.166, in a range of 0.166-0.332, in a range of 0.332-0.498, in a range of 0.498-0.664, in a range of 0.664-0.830, and in a range of 0.830-1 is shown.

The MCI accuracy is obtained by the number of MCIs/(the number of MCIs+the number of NDCs) in the NIRS index range. For example, when the measurement data of the MCI subjects is calculated using the regression model of ID1, the number of MCIs in a NIRS index range of 0-0.166 is counted as 1 when the NIRS index of the measurement data is determined to be 0.1. Similarly, when the measurement data of the NDC subjects is calculated using the regression model of ID1, the number of NDCs in a NIRS index range of 0-0.166 is counted as 1 when the NIRS index of the measurement data is determined to be 0.1. In this manner, the MCI accuracy in each NIRS index range is obtained by classifying the measurement data of the thirty-eight subjects into MCI and NDC in each NIRS index range. This operation is performed for the five regression models having a high accuracy rate such that the graphs shown in FIGS. 7 to 11 are obtained.

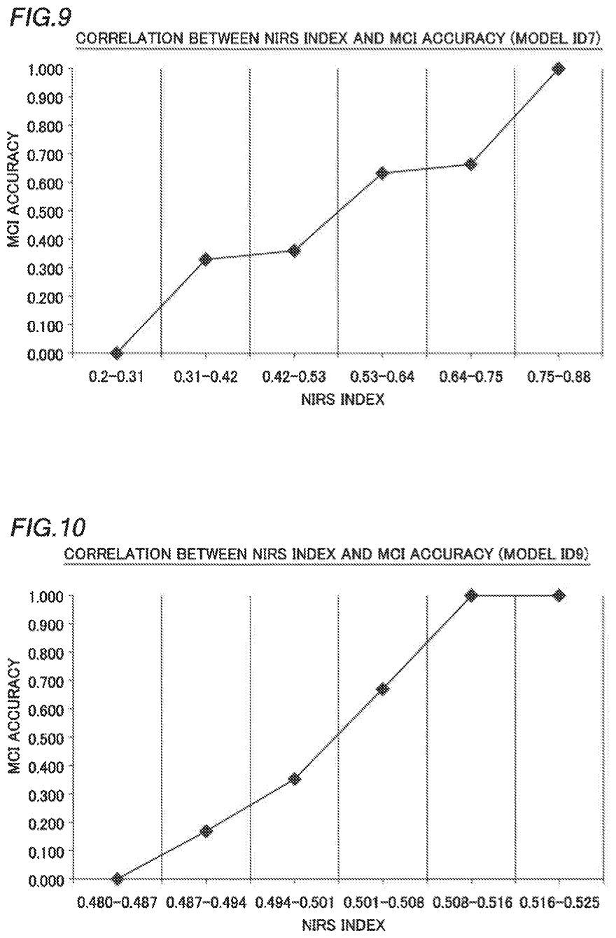

When the graphs shown in FIGS. 7 to 11 are compared, it has been found that in the regression model of ID7 (see FIG. 9), there is a proportional relationship between the NIRS index, which is the index indicating the change in the biological activity (brain activity) related to the cognitive function of the subject P, and the MCI accuracy, which is the index indicating the cognitive function of the subject P. It has been found that the regression model of ID7 shows a high correlation between the NIRS index, which is the index indicating the change in the biological activity (brain activity) related to the cognitive function of the subject P, and the MCI accuracy, which is the index indicating the cognitive function of the subject P. From this, it has been found that the cognitive function of the subject P can be indexed based on the NIRS index, which is the index indicating the change in the biological activity (brain activity) related to the cognitive function of the subject P.

Also, for the five regression models of IDs 1, 4, 7, 9, and 12, a significant difference test was performed between an NDC group and an MCI group based on evaluation data (measurement data of thirteen subjects). The results of the significant difference test are shown as graphs in FIGS. 12 to 16. As shown in FIGS. 12 to 16, a significant difference p between the NDC group and the MCI group was 0.354 for the regression model of ID1, 0.265 for the regression model of ID4, 0.026 for the regression model of ID7, 0.776 for the regression model of ID9, and 0.937 for the regression model of ID12. As described above, it has been found that only the significant difference p of the regression model of ID7 is 0.05 or less (5% or less), which is a significance level, and only the regression model of ID7 can significantly distinguish the NDC group from the MCI group.

### Advantages of this Embodiment

According to this embodiment, the following advantages are obtained.

According to this embodiment, as described above, the method for indexing cognitive function includes the step of acquiring the index indicating the cognitive function of the subject P from the measurement data D of the subject P using the model M constructed in advance based on the measurement data D of the group of non-demented persons acquired in advance and the measurement data D of the group of persons with mild cognitive impairment acquired in advance. Accordingly, when the subject P who is a non-demented person, the subject P who is a person with mild cognitive impairment, the subject P who is anxious about his or her cognitive function, etc. perform interventions for dementia prevention (such as exercise) to prevent dementia, the index indicating the cognitive function of the subject P before and after the interventions for dementia prevention can be acquired and compared, and thus the subject P can know a change in his or her cognitive function before and after the interventions for dementia prevention (i.e., the effects of the interventions for dementia prevention). In addition, when the subject P continuously performs the interventions for dementia prevention, the change in the cognitive function of the subject P due to the continuous interventions for dementia prevention (i.e., the effects of the interventions for dementia prevention) can be shown to the subject P, and thus the subject P can know the change in the cognitive function due to the continuous interventions for dementia prevention. As described above, the subject P can know the effects of the interventions for dementia prevention, and thus the subject P can be effectively motivated to voluntarily and continuously perform the interventions for dementia prevention. The method for indexing cognitive function according to this embodiment can be suitably implemented in a welfare facility or a sports gym, for example.

According to this embodiment, as described above, the step of acquiring the index indicating the cognitive function of the subject P includes the step of acquiring a numerical value as the index indicating the cognitive function of the subject P from the measurement data D of the subject P using the model M. Accordingly, the effects of the interventions for dementia prevention can be more clearly measured.

According to this embodiment, as described above, the step of giving a work includes the step of giving a plurality of works with different degrees of difficulty to the subject P. Furthermore, the step of acquiring the index indicating the cognitive function of the subject P includes the step of acquiring the index indicating the cognitive function of the subject P from the measurement data D of the subject P using the model M constructed based on the measurement data D of the group of non-demented persons given the same work as that given to the subject P and the measurement data D of the group of persons with mild cognitive impairment given the same work as that given to the subject P. Accordingly, the plurality of works with different degrees of difficulty are given such that it is possible to measure whether or not the subject P has adapted to a difficult work. Consequently, the change in the biological activity related to the cognitive function of the subject P can be more clearly caused, and thus the index indicating the cognitive function of the subject P can be more accurately acquired.

According to this embodiment, as described above, the step of acquiring the index indicating the cognitive function of the subject P includes the step of acquiring the index indicating the cognitive function of the subject P from the measurement data D of the subject P using the model M constructed using the same type of feature amount as that of the measurement data D of the subject P, the feature amount being the difference or ratio between the values of the measurement data D of the subject P in the plurality of works with different degrees of difficulty. Accordingly, the difference or ratio between the values of the measurement data D of the subject P is used as the feature amount such that the influence of a parameter unique to the subject P, which occurs for each subject P due to the shape of the measurement site of the subject P, for example, can be eliminated. Therefore, the index indicating the cognitive function of the subject P can be accurately acquired regardless of the subject P.

According to this embodiment, as described above, the step of acquiring the index indicating the cognitive function of the subject P includes the step of acquiring the index indicating the cognitive function of the subject P from the measurement data D of the subject P using the model M constructed using the same type of feature amount as that of the measurement data D of the subject P, the feature amount being the average value of the waveform W of the measurement data D of the subject P, the value indicating the area centroid of the waveform W of the measurement data D of the subject P, or the value indicating the slope of the waveform W of the measurement data D of the subject P. Accordingly, the average value of the waveform W of the measurement data D of the subject P, the value indicating the area centroid of the waveform W of the measurement data D of the subject P, or the value indicating the slope of the waveform W of the measurement data D of the subject P, which is a feature amount strongly related to the cognitive function, can be used as the feature amount, and thus the index indicating the cognitive function of the subject P can be accurately acquired.

According to this embodiment, as described above, the step of acquiring the index indicating the cognitive function of the subject P includes the step of acquiring the index indicating the cognitive function of the subject P from the measurement data D of the subject P using the model M constructed using the same type of feature amount as that of the measurement data D of the subject P, the feature amount being the change in the amount of oxygenated hemoglobin, the change in the amount of deoxygenated hemoglobin, or the change in the total amount of hemoglobin. Accordingly, the change in the amount of oxygenated hemoglobin, the change in the amount of deoxygenated hemoglobin, or the change in the total amount of hemoglobin that easily occurs with the work for inducing the biological activity related to the cognitive function can be used as the feature amount, and thus the index indicating the cognitive function of the subject P can be accurately acquired.

According to this embodiment, as described above, the step of giving a work includes the step of giving, as a task, at least one of sensory stimulation, calculation, memorization, imagination, and spatial recognition to the subject P. Furthermore, the step of acquiring the index indicating the cognitive function of the subject P includes the step of acquiring the index indicating the cognitive function of the subject P from the measurement data D of the subject P using the model M constructed based on the measurement data D of the group of non-demented persons given the same work as that given to the subject P and the measurement data D of the group of persons with mild cognitive impairment given the same work as that given to the subject P. Accordingly, at least one of sensory stimulation, calculation, memorization, imagination, and spatial recognition, which is a task suitable for inducing the biological activity related to the cognitive function, can be given to the subject P as a task, and thus the measurement data D can be acquired by reliably measuring the change in the biological activity related to the cognitive function of the subject P.

According to this embodiment, as described above, the step of giving a work includes the step of giving the work for inducing the brain activity related to the cognitive function to the subject P. Furthermore, the step of acquiring the measurement data D includes the step of acquiring the measurement data D by measuring the change in the cerebral blood flow of the subject P when the work is given to the subject P. Accordingly, the measurement data D can be acquired by measuring the change in the cerebral blood flow suitable for measuring the cognitive function, and thus the index indicating the cognitive function of the subject P can be accurately acquired.

According to this embodiment, as described above, the step of acquiring the index indicating the cognitive function of the subject P includes the step of acquiring the index indicating the cognitive function of the subject P from the measurement data D of the subject P using the model M constructed by the regression model. Accordingly, the index indicating the cognitive function of the subject P can be acquired from the measurement data D of the subject P using the model M that accurately represents the correlation between the non-demented persons and the persons with mild cognitive impairment, and thus the index indicating the cognitive function of the subject P can be accurately acquired.

### MODIFIED EXAMPLES

The embodiment disclosed this time must be considered as illustrative in all points and not restrictive. The scope of the present invention is not shown by the above description of the embodiment but by the scope of claims for patent, and all modifications (modified examples) within the meaning and scope equivalent to the scope of claims for patent are further included.

For example, while the work for inducing the brain activity related to the cognitive function is given to the subject in the aforementioned embodiment, the present invention is not limited to this. In the present invention, a work for inducing biological activity other than the brain activity related to the cognitive function may alternatively be given to the subject. In this case, a change in biological activity other than the change in the cerebral blood flow of the subject may be measured when the work is given to the subject. For example, a change in the gaze of the subject or a change in the behavior of the subject related to his or her cognitive function may be measured when the work is given to the subject.

While a numerical value is acquired as the index indicating the cognitive function of the subject in the aforementioned embodiment, the present invention is not limited to this. In the present invention, a value other than a numerical value may alternatively be acquired as the index indicating the cognitive function of the subject.

While a plurality of works with different degrees of difficulty are given to the subject in the aforementioned embodiment, the present invention is not limited to this. In the present invention, a plurality of works with the same degree of difficulty may alternatively be given to the subject. Alternatively, only a single work may be given to the subject.

While the difference or ratio between the values of the measurement data of the subject in the plurality of works with different degrees of difficulty is used as the feature amount in the aforementioned embodiment, the present invention is not limited to this. In the present invention, even when the plurality of works with different degrees of difficulty are given to the subject, the difference or ratio between the values of the measurement data of the subjects in the plurality of works with different degrees of difficulty does not necessarily have to be used as the feature amount.

While the average value of the waveform of the measurement data, the value indicating the area centroid of the waveform of the measurement data, or the value indicating the slope of the waveform is used as the feature amount in the aforementioned embodiment, the present invention is not limited to this. In the present invention, a value indicating the feature of the waveform other than the average value of the waveform of the measurement data, the value indicating the area centroid of the waveform of the measurement data, or the value indicating the slope of the waveform may alternatively be used as the feature amount.

While the change in the amount of oxygenated hemoglobin, the change in the amount of deoxygenated hemoglobin, or the change in the total amount of hemoglobin is used as the feature amount in the aforementioned embodiment, the present invention is not limited to this. In the present invention, a feature amount other than the change in the amount of oxygenated hemoglobin, the change in the amount of deoxygenated hemoglobin, or the change in the total amount of hemoglobin may alternatively be used as the feature amount. For example, the accuracy rate of the task or the performance of the task may be used as the feature amount.

While the model is constructed by the regression model based on logistic regression in the aforementioned embodiment, the present invention is not limited to this. In the present invention, the model may alternatively be constructed by other than the regression model based on logistic regression.

While when the task to be given to the subject is sensory stimulation, cold sensory stimulation is given to the subject in the aforementioned embodiment, the present invention is not limited to this. In the present invention, as long as the task is sensory stimulation given to the sensory organ of the subject, sensory stimulation other than the cold sensory stimulation may alternatively be given to the subject.

While when the task to be given to the subject is calculation, the calculation problem of subtraction is given to the subject in the aforementioned embodiment, the present invention is not limited to this. In the present invention, any calculation problem of four arithmetic operations may alternatively be given to the subject.

What is claimed is:

1. A method for indexing cognitive function, comprising:
giving, to a subject, a work for inducing biological activity related to cognitive function;
acquiring measurement data by measuring a change in the biological activity related to the cognitive function of the subject when the work is given to the subject; and
acquiring an index indicating the cognitive function of the subject from the measurement data of the subject using a model constructed in advance based on the measurement data of a group of non-demented persons acquired in advance and the measurement data of a group of persons with mild cognitive impairment acquired in advance.

2. The method for indexing cognitive function according to claim 1, wherein the acquiring of the index indicating the cognitive function of the subject includes acquiring, as the index indicating the cognitive function of the subject, a numerical value from the measurement data of the subject using the model.

3. The method for indexing cognitive function according to claim 1, wherein
the giving of the work includes giving a plurality of works with different degrees of difficulty to the subject; and
the acquiring of the index indicating the cognitive function of the subject includes acquiring the index indicating the cognitive function of the subject from the measurement data of the subject using the model constructed based on the measurement data of the group of the non-demented persons given a same work as that given to the subject and the measurement data of the group of the persons with mild cognitive impairment given the same work as that given to the subject.

4. The method for indexing cognitive function according to claim 3, wherein the acquiring of the index indicating the cognitive function of the subject includes acquiring the index indicating the cognitive function of the subject from the measurement data of the subject using the model constructed using a same type of feature amount as that of the measurement data of the subject, the feature amount being a difference or ratio between values of the measurement data of the subject in the plurality of works with the different degrees of difficulty.

5. The method for indexing cognitive function according to claim 1, wherein the acquiring of the index indicating the cognitive function of the subject includes acquiring the index indicating the cognitive function of the subject from the measurement data of the subject using the model constructed using a same type of feature amount as that of the measurement data of the subject, the feature amount being an average value of a waveform of the measurement data of the subject, a value indicating an area centroid of the waveform of the measurement data of the subject, or a value indicating a slope of the waveform of the measurement data of the subject.

6. The method for indexing cognitive function according to claim 1, wherein the acquiring of the index indicating the cognitive function of the subject includes acquiring the index indicating the cognitive function of the subject from the measurement data of the subject using the model constructed using a same type of feature amount as that of the measurement data of the subject, the feature amount being a change in an amount of oxygenated hemoglobin, a change in an amount of deoxygenated hemoglobin, or a change in a total amount of hemoglobin.

7. The method for indexing cognitive function according to claim 1, wherein
the giving of the work includes giving, as a task, at least one of sensory stimulation, calculation, memorization, imagination, and spatial recognition to the subject; and
the acquiring of the index indicating the cognitive function of the subject includes acquiring the index indicating the cognitive function of the subject from the measurement data of the subject using the model constructed based on the measurement data of the group of the non-demented persons given a same work as that given to the subject and the measurement data of the group of the persons with mild cognitive impairment given the same work as that given to the subject.

8. The method for indexing cognitive function according to claim 1, wherein
the giving of the work includes giving the work for inducing brain activity related to the cognitive function to the subject; and
the acquiring of the measurement data includes acquiring the measurement data by measuring a change in a cerebral blood flow of the subject when the work is given to the subject.

9. The method for indexing cognitive function according to claim 1, wherein the acquiring of the index indicating the cognitive function of the subject includes acquiring the index indicating the cognitive function of the subject from the measurement data of the subject using the model including a regression model.
